(19) United States
(12) Patent Application Publication
(10) Pub. No.: US 2026/0268176 A1
(43) Pub. Date: Sep. 10, 2026

(54) BLENDING RATIO DETERMINATION DEVICE, BLENDING RATIO DETERMINATION METHOD, AND RECORDING MEDIUM

(71) Applicant: NEC Corporation, Tokyo (JP)

(72) Inventors: Ayako HOSHINO, Tokyo (JP); Masaru INABA, Tokyo (JP)

(73) Assignee: NEC Corporation, Tokyo (JP)

(21) Appl. No.: 19/165,637

(22) PCT Filed: Mar. 28, 2023
(86) PCT No.: PCT/JP2023/012421

§ 371 Date: Sep. 16, 2025

(51) Int. Cl. G06N 5/04; G06Q 30/0283

(52) U.S. Cl. CPC G06N 5/04; G06Q 30/0283

(57) ABSTRACT

In a blending ratio determination device, a training means performs training using compatibility scores between materials whose compatibility is known, and generates a prediction model that predicts compatibility scores between materials whose compatibility is unknown. A compatibility prediction means generates compatibility score information including compatibility scores between the materials whose compatibility is unknown, by using the prediction model. A problem generation means generates an optimization problem including compatibility scores between a plurality of materials to be blended in an objective function. A blending ratio determination means determines the blending ratios of the plurality of materials by solving the optimization problem. The blending ratio determination device can, for example, support decision making in determining the blending ratios.

### TECHNICAL FIELD

The present disclosure relates to a technology for determining blending a ratio of materials.

### BACKGROUND ART

In a case of creating foods or feeds, a method for determining appropriate blending of materials has been proposed. For instance, Patent Document 1 describes a blending design method that uses linear programming to determine the blending of feed for livestock and the like.

### PRECEDING TECHNICAL REFERENCES

### Patent Document

Patent Document 1: Japanese Laid-open Patent Publication No. H8-201167

### SUMMARY

### Problem to be Solved by the Invention

The blending design method of Patent Document 1 determines blending ratios so as to minimize blending costs, and the combination of materials to be used is not necessarily appropriate.

It is one object of the present disclosure to determine appropriate blending ratios by considering compatibility between materials.

### Means for Solving the Problem

According to an example aspect of the present disclosure, there is provided blending ratio determination device including:
a training means configured to perform training by using a compatibility score between materials whose compatibility is known, and generate a prediction model that predicts a compatibility score between materials whose compatibility is unknown;
a compatibility prediction means configured to generate, by using the prediction model, compatibility score information including the compatibility score between the materials whose compatibility is unknown;
a problem generation means configured to generate an optimization problem including compatibility scores between a plurality of materials to be blended in an objective function; and
a blending ratio determination means configured to determine blending ratios of the plurality of materials to be blended by solving the optimization problem.

According to another example aspect of the present disclosure, there is provided a blending ratio determination method executed by a computer, the blending ratio determination method including:
performing training by using a compatibility score between materials whose compatibility is known, and generating a prediction model that predicts a compatibility score between materials whose compatibility is unknown;
generating, by using the prediction model, compatibility score information including the compatibility score between the materials whose compatibility is unknown;
generating an optimization problem including compatibility scores between a plurality of materials to be blended in an objective function; and
determining blending ratios of the plurality of materials to be blended by solving the optimization problem.

According to a further example aspect of the present disclosure, there is provided a recording medium storing a program, the program causing a computer to perform a process including:
performing training by using a compatibility score between materials whose compatibility is known, and generating a prediction model that predicts a compatibility score between materials whose compatibility is unknown;
generating, by using the prediction model, compatibility score information including the compatibility score between the materials whose compatibility is unknown;
generating an optimization problem including compatibility scores between a plurality of materials to be blended in an objective function; and
determining blending ratios of the plurality of materials to be blended by solving the optimization problem.

### BRIEF DESCRIPTION OF THE DRAWINGS

FIG. 1 illustrates an outline of a blending ratio determination device according to a first example embodiment.

FIG. 2 is a block diagram illustrating a hardware configuration of the blending ratio determination device.

FIG. 3 is a block diagram illustrating a functional configuration of the blending ratio determination device.

FIG. 4A and FIG. 4B illustrate examples of a compatibility table and graph link data.

FIG. 5 illustrates an example of the compatibility table created by the compatibility prediction unit.

FIG. 6 illustrates an example of a cost table.

FIG. 7 illustrates an example of an optimization problem.

FIG. 8 is a flowchart of a blending ratio determination process.

FIG. 9 illustrates an example of a display of the blending ratios determined by the blending ratio determination device.

FIG. 10 is a block diagram illustrating a configuration of a blending ratio determination device according to a second example embodiment.

FIG. 11 is a flowchart of process by the blending ratio determination device according to the second example embodiment.

### EXAMPLE EMBODIMENTS

In the following, example embodiments will be described with reference to the accompanying drawings.

### First Example Embodiment

### [Overall Configuration]

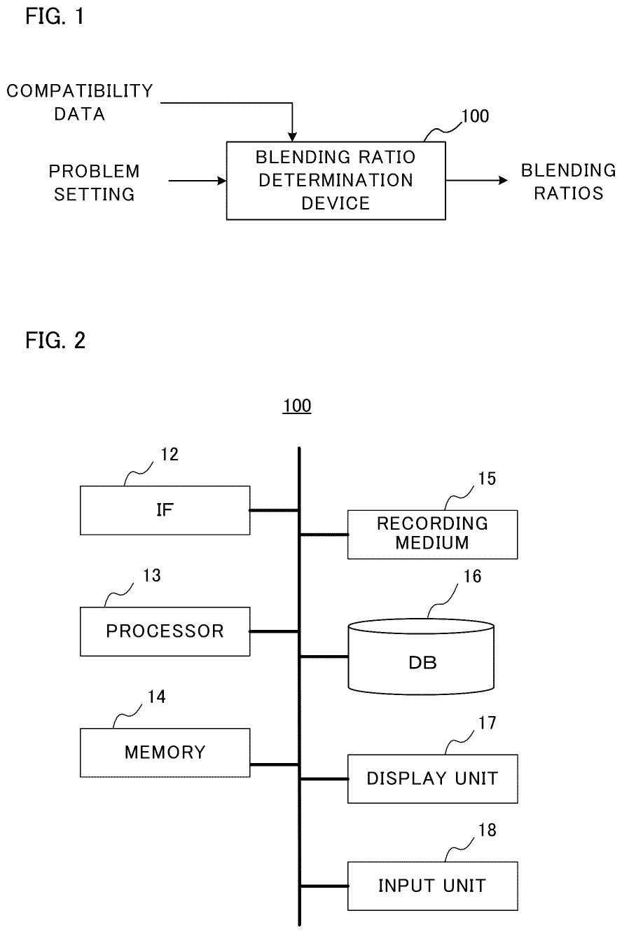

FIG. 1 illustrates an outline of a blending ratio determination device 100 according to a first example embodiment. The blending ratio determination device 100 is a device that determines appropriate blending ratios of a plurality of materials. In the following description, the blending ratio determination device 100 is assumed to determine blending ratios in a case of blending the plurality of materials (food materials) to create food, but the present disclosure is not limited to this application. For instance, the present disclosure can be applied to cases where various substances are created by blending the plurality of materials, such as blending feed for livestock, blending dyes, blending metal materials, and the like.

Compatibility data and problem setting are input to the blending ratio determination device 100. The compatibility data are data indicating compatibility between the plurality of materials, specifically, compatibility scores indicating quality of compatibility. Here, the compatibility data input to the blending ratio determination device 100 are compatibility data between materials whose compatibilities have been known. That is, the compatibility scores between materials, whose good or poor compatibility has been established through prior experiments or existing knowledge, are input as the compatibility data. The blending ratio determination device 100 performs training based on the input compatibility data to create a prediction model that predicts each compatibility between the materials whose compatibility is unknown. Furthermore, the blending ratio determination device 100 uses the prediction model to create a compatibility table illustrating each compatibility score between the plurality of materials that can be used for blending.

Next, the blending ratio determination device 100 receives input of the problem setting. The problem setting indicates conditions for determining optimum blending ratios of the plurality of materials. Specifically, the problem setting includes the number of materials to be blended, specification of the materials to be blended, a condition for necessary nutritional values, and a condition for necessary costs. The blending ratio determination device 100 calculates the optimum blending ratios that satisfies the input problem setting by using the compatibility table, and outputs the optimum blending ratios.

### [Hardware Configuration]

FIG. 2 is a block diagram illustrating a hardware configuration of the blending ratio determination device 100. As illustrated, the blending ratio determination device 100 includes an interface (IF) 12, a processor 13, a memory 14, a recording medium 15, a database (DB) 16, a display unit 17, and an input unit 18.

The IF 12 acquires the compatibility data and the problem setting. The compatibility data may be input by a user, or pre-prepared compatibility data may be read from the recording medium 15 or the like. The problem setting is basically input by the user. Furthermore, the IF 12 outputs the blending ratios determined by the blending ratio determination device 100 to the display unit 17 or an external device.

The processor 13 is a computer such as a CPU (Central Processing Unit), and controls the entire blending ratio determination device 100 by executing programs prepared in advance. As the processor 13, a CPU, a GPU (Graphics Processing Unit), a DSP (Digital Signal Processor), an MPU (Micro Processing Unit), an FPU (Floating Point number Processing Unit), a PPU (Physics Processing Unit), a TPU (Tensor Processing Unit), a quantum processor, a microcontroller, or a combination thereof can be used. The processor 13 executes a blending ratio determination process, which will be described later.

The memory 14 is formed by a ROM (Read Only Memory), a RAM (Random Access Memory), and the like. The memory 14 stores various programs executed by the processor 13. The memory 14 is also used as a working memory during executions of various processes by the processor 13.

The recording medium 15 is a non-volatile and non-transitory recording medium such as a disk-shaped recording medium or a semiconductor memory, and is configured to be detachable from the blending ratio determination device 100. The recording medium 15 records various programs executed by the processor 13. In a case where the blending ratio determination device 100 executes various processes, the programs recorded in the recording medium 15 are loaded into the memory 14 and executed by the processor 13.

The DB 16 stores the compatibility data input via the IF 12. The blending ratios calculated by the blending ratio determination device 100 is stored in the DB 16 as needed.

The display unit 17 is formed by, for instance, a liquid crystal display. The blending ratios determined by the blending ratio determination device 100 for the input problem setting is displayed on the display unit 17. The input unit 18 includes, for instance, a keyboard and a mouse. The user inputs the compatibility data and the problem setting to the blending ratio determination device 100 using the input unit 18.

### [Functional Configuration]

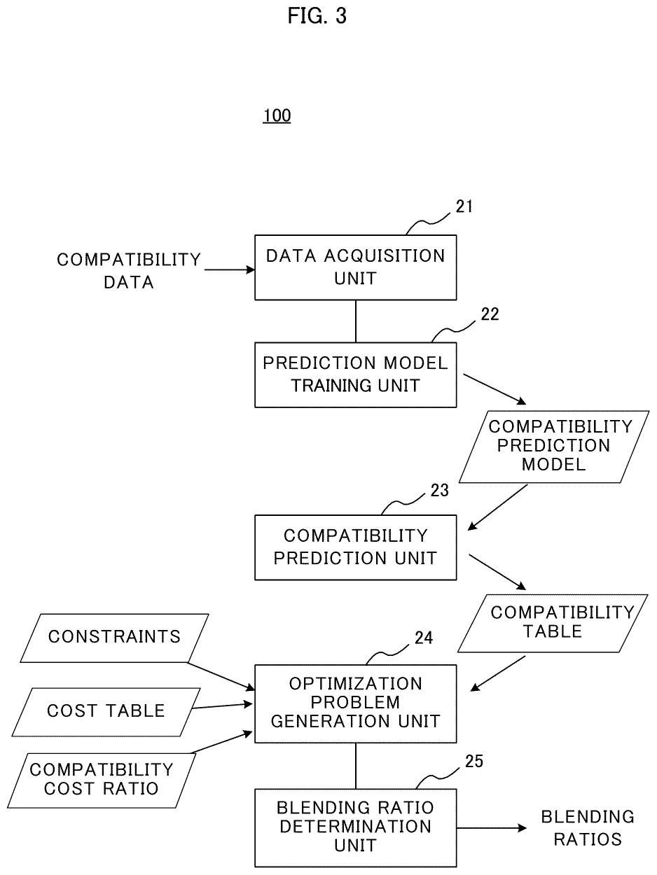

FIG. 3 is a block diagram illustrating a functional configuration of the blending ratio determination device 100. The blending ratio determination device 100 functionally includes a data acquisition unit 21, a prediction model training unit 22, a compatibility prediction unit 23, an optimization problem generation unit 24, and a blending ratio determination unit 25.

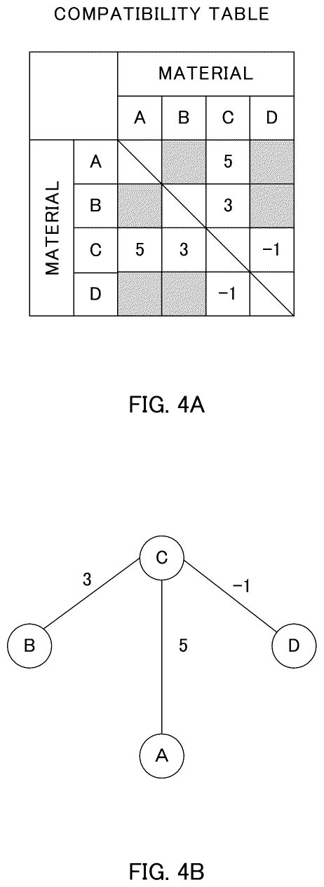

The data acquisition unit 21 acquires compatibility data between the materials whose compatibility is known. The compatibility data are given as two materials and the compatibility score indicating the compatibility between those materials. The data acquisition unit 21 creates the compatibility table based on the input compatibility data. FIG. 4A illustrates an example of the compatibility table. The compatibility table is an example of compatibility score information, and is a table illustrating respective compatibility scores between the plurality of materials. In the example in FIG. 4A, the compatibility table illustrates the compatibility scores between four materials A to D. That is, in the example in FIG. 4A, compatibility data are input indicating that the compatibility score between the material A and the material C is “5”, the compatibility score between the material B and the material C is “3”, and the compatibility score between the material C and the material D is “−1”. Note that a positive compatibility score indicates good compatibility, and a negative compatibility score indicates poor compatibility. On the other hand, the gray boxes in FIG. 4A correspond to the compatibility scores between the materials whose compatibility is unknown and are blank. The data acquisition unit 21 outputs the compatibility table created based on the input compatibility data to the prediction model training unit 22.

The prediction model training unit 22 learns the compatibility between the materials using the input compatibility table and generates the prediction model. The prediction model training unit 22 generates the prediction model that performs graph link prediction. Specifically, the prediction model training unit 22 creates graph link data based on the input compatibility table. The graph link data are data that represent respective relationships between the materials with each material as a node and links (edges) connecting the node. FIG. 4B illustrates the graph link data corresponding to the compatibility table in FIG. 4A. The materials A to D are set as nodes, and the links between the nodes indicate the compatibility scores illustrating the relationships (compatibilities) between the materials.

The prediction model training unit 22 generates the graph link data based on the compatibility data input to the data acquisition unit 21, and generates the prediction model by performing graph link learning using the generated graph link data. The graph link learning is a method of extracting rules indicating the relationship between two nodes and their nodes from the relationships between each node and link included in the graph link data, and generating the prediction model using the extracted rules. In this example embodiment, the prediction model training unit 22 extracts the rules indicating the compatibility between two materials based on the compatibility data input to the data acquisition unit 21, that is, the graph link data indicating the compatibility scores between the materials whose compatibility is known, and generates the prediction model that predicts the compatibility scores between two materials whose compatibility is unknown. That is, the prediction model training unit 22 generates the prediction model that predicts the compatibility scores that fit into the gray boxes in FIG. 4A based on the compatibility table illustrated in FIG. 4A. The prediction model training unit 22 outputs the generated prediction model to the compatibility prediction unit 23.

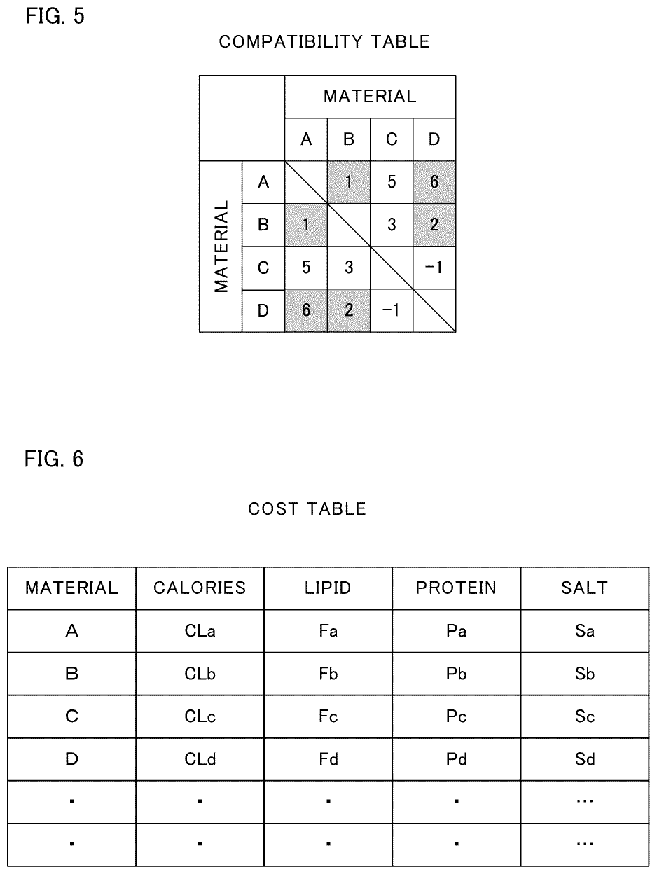

The compatibility prediction unit 23 uses the prediction model input from the prediction model training unit 22 to predict the compatibility scores between the materials whose compatibility is unknown, and creates the compatibility table that includes compatibility scores between the materials whose compatibility is unknown. FIG. 5 illustrates an example of the compatibility table created by the compatibility prediction unit 23. As can be seen by comparing the compatibility table with that in FIG. 4A, in the compatibility table of FIG. 5, the compatibility scores between the materials whose compatibilities are unknown are predicted by the prediction model, and are input into the corresponding boxes. In this way, in the present example embodiment, by performing the graph link prediction using the prediction model learned based on known compatibility data, it is possible to create the compatibility table including compatibility data for all materials. The compatibility prediction unit 23 outputs the created compatibility table to the optimization problem generation unit 24.

The optimization problem generation unit 24 generates an optimization problem for determining the optimum blending ratios of the materials. In addition to the compatibility table, a constraint condition, a cost table, and a compatibility cost ratio are input to the optimization problem generation unit 24. The constraint condition, the cost table, and the compatibility cost ratio are examples of the problem setting described above, and are basically input by the user who determines the blending ratios.

The optimization problem generation unit 24 first sets an objective function f of the optimization problem as follows.

$$f = ( total nutritional value score ) + α ( total compatibility score ) \qquad ( 1 )$$

Here, “α” is the compatibility cost ratio, which is a parameter indicating a ratio of a total nutritional value score to a total compatibility score in the objective function f.

The “total nutritional value score” is a sum of the nutritional value scores obtained from the plurality of materials to be blended. The nutritional value score can be calculated for each nutritional component such as protein, vitamins, and calories, and is calculated based on the cost table. FIG. 6 illustrates an example of the cost table. The cost table is a table that indicates the nutritional value per unit weight for each of the plurality of materials. In the example in FIG. 6, for each material, calories, fat, protein, and salt are defined as nutritional components. The optimization problem generation unit 24 refers to the cost table and calculates the nutritional value score for the target nutritional components (e.g., calories, protein, etc.) for each of the plurality of materials to be blended. Specifically, the optimization problem generation unit 24 calculates the blending amount for each of the plurality of materials to be blended based on the blending ratios, calculates the nutritional value score for each material by referring to the cost table, and sums the nutritional value scores to calculate the total nutritional value score. Note that in a case of calculating the total nutritional value score to be used in the objective function, it is preferable to sum the nutrient value scores of the nutritional components that are desirable in large amounts, such as proteins, vitamins, and the like, as positive values, and it is preferable to sum the nutrient value scores of the nutritional components that are undesirable in large amounts, such as calories, as negative values summing the scores of less desirable nutrients, such as calories and the like, as negative values. For instance, in a case of using protein and calories as the nutritional components, the total nutritional value score may be obtained by subtracting the total calories of each material from the total protein of each material.

The “total compatibility score” is calculated as the sum of compatibility scores between the plurality of materials to be blended. If n types of materials are to be blended and the blending ratios of materials are represented by x₁, x₂, . . . , xₙ, then the total compatibility score is obtained by the following formula.

$$( Total compatibility score ) = \sum {x}_{i} \times {x}_{j} \times {r\_x}_{i} {x}_{j} \qquad ( 2 )$$

where r_xᵢxⱼ is the compatibility score between material i and material j.

As an example, in a case of using the materials A to C, the total compatibility score is obtained by the following formula.

$$( Total compatibility score ) = ( blending ratio of material A ) \times ( blending ratio of material B ) \times ( compatibility score for materials A and B ) + ( blending ratio of material B ) \times ( blending ratio of material C ) \times ( compatibility score for materials B and C ) + ( blending ratio of material C ) \times ( blending ratio of material A ) \times ( compatibility score for materials C and A )$$

In this way, by including the total compatibility score, which is calculated based on the compatibility scores between the materials to be used, in the objective function of the optimization problem, it is possible to obtain the optimum blending ratios in consideration of the compatibility between the materials.

Furthermore, the optimization problem generation unit 24 generates the optimization problem using the constraint condition input by the user. Here, the constraint condition can include, for instance, the number of materials to be used, the specification of materials to be used, and the conditions related to the nutritional components such as nutritional values.

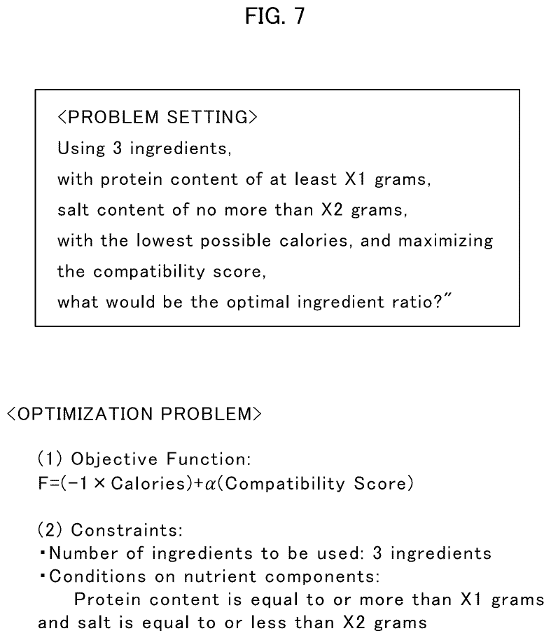

FIG. 7 illustrates an example of the optimization problem. Now, assume that the user inputs the problem setting illustrated in FIG. 7 to the blending ratio determination device 100. In this case, the optimization problem generation unit 24 generates the following objective function to minimize calories and maximize the compatibility score.

$$f = ( ( - 1 ) \times calories ) + α ( total compatibility score )$$

Here, the value input by the user is used for the compatibility cost ratio α.

Furthermore, the optimization problem generation unit 24 determines the following constraint conditions based on the problem setting.
Number of materials to be used: 3
Conditions on nutrient components: Protein content is equal to or more than X1 grams and salt is equal to or less than X2 grams

In this way, the optimization problem generation unit 24 generates the optimization problem including the objective function and the constraint conditions based on the problem setting input by the user, and outputs the optimization problem to the blending ratio determination unit 25.

The blending ratio determination unit 25 outputs, as optimum blending ratios, solutions obtained by solving the input optimization problem. The blending ratio determination unit 25 may determine the blending ratios by using, for example, a solver of linear programming. In this case, as indicated in Expression (2), since the total compatibility score included in the objective function is a quadratic expression including the two variables xᵢ and xⱼ, the blending ratio determination unit 25 may apply the linear programming by approximating Expression (2) to a linear expression by using a min-max method. Alternatively, the blending ratio determination unit 25 may determine the blending ratios by using a method such as quadratic programming that can handle an objective function of a quadratic expression. In the present example embodiment, the method by which the blending ratio determination unit 25 derives the optimum solution from the optimization problem is not limited to a specific method.

In the above configuration, the prediction model training unit 22 is an example of training means, the compatibility prediction unit 23 is an example of compatibility prediction means, the optimization problem generation unit 24 is an example of problem generation means, and the blending ratio determination unit 25 is an example of blending ratio determination means.

### [Blending Ratio Determination Process]

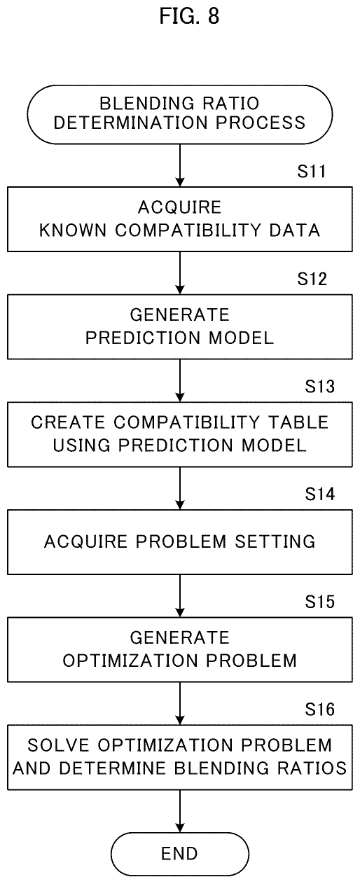

FIG. 8 is a flowchart of the blending ratio determination process. This process is realized by the processor 13 illustrated in FIG. 2 executing a corresponding program prepared in advanced and operating as each element illustrated in FIG. 3.

First, the data acquisition unit 21 acquires known compatibility data (step S11). Next, the prediction model training unit 22 generates the graph link data using the compatibility data, and learns the compatibility between the materials using the graph link data to generate the prediction model that predicts the compatibility between unknown materials (step S12). Next, the compatibility prediction unit 23 predicts compatibility between unknown materials not included in the compatibility data by using the generated prediction model, and creates a compatibility table (step S13).

Next, the optimization problem generation unit 24 acquires the compatibility table from the compatibility prediction unit 23, and also acquires the problem setting input by the user, specifically the constraint condition, the cost table, the compatibility cost ratio, and the like (step S14). Next, the optimization problem generation unit 24 generates the optimization problem including the objective function and the constraint condition based on the compatibility table, the constraint condition, the compatibility cost ratio, and the cost table, which have been acquired (step S15). Note that the objective function includes the total compatibility score, which is the sum of the compatibility scores between the plurality of materials to be used. Next, the blending ratio determination unit 25 obtains the solution to the optimization problem using the linear programming, the quadratic programming, or other methods, and outputs the blending ratios (step S16). After that, the blending ratio determination process is terminated.

### [Display Example]

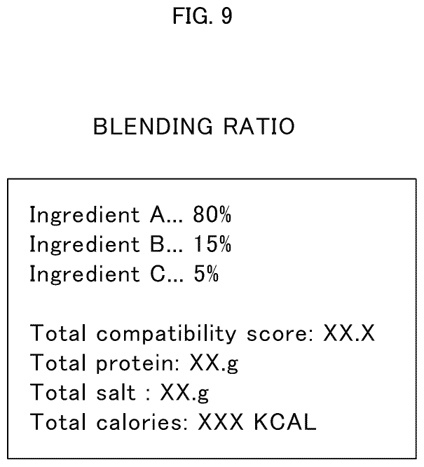

FIG. 9 illustrates a display example of the blending ratios determined by the blending ratio determination device 100. The display example in FIG. 9 illustrates the determined blending ratios on the display unit 17. This example illustrates a result of determining the blending ratios of three materials (ingredients) A to C based on the problem setting illustrated in FIG. 7. Specifically, the blending ratios of ingredients A to C are obtained as “80%”, “15%”, and “5%”, respectively. In addition, totals of nutritional values (total protein, total salt, and total calories) included in the problem setting are illustrated so that the user can easily confirm whether the obtained blending ratios satisfy the problem setting. Furthermore, a total of compatibility scores between the used ingredients is displayed.

### [Application Example]

The above blending ratio determination device 100 may be applied to the development of health foods and supplements for healthcare. Some materials used for the health foods, the supplements, and the like also have known compatibility. For instance, compatibility data can be generated based on known knowledge such as “a certain ingredient F includes a large amount of nutrients G, and absorption of the nutrients G is promoted when the ingredient F is taken together with a material H”, experimental results, and the like, and a compatibility table of a plurality of ingredients can be created. Then, by generating an optimization problem using factors such as the target intake amounts of nutrients, constraint condition on calories and cost, and determining the optimum blending ratios, it is possible to develop the health foods, the supplements, and the like that help the body efficiently absorb desired nutrients.

### Second Example Embodiment

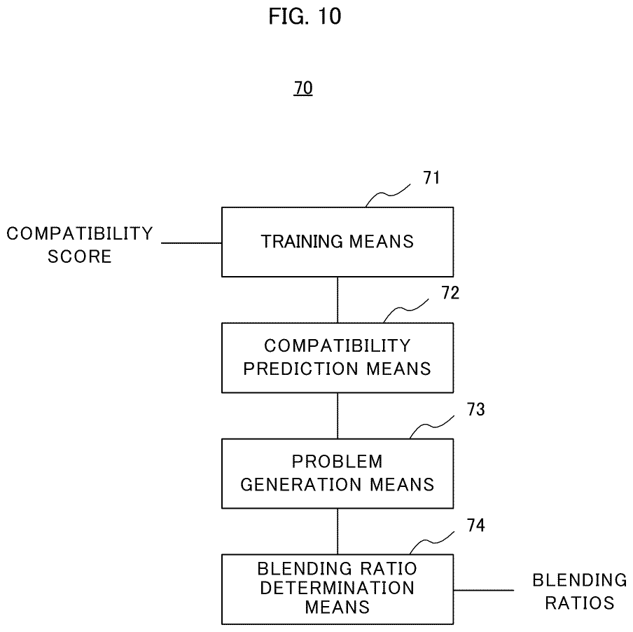

FIG. 10 is a block diagram illustrating a configuration of a blending ratio determination device according to a second example embodiment. A blending ratio determination device 70 according to the second example embodiment includes a training means 71, a compatibility prediction means 72, a problem generation means 73, and a blending ratio determination means 74.

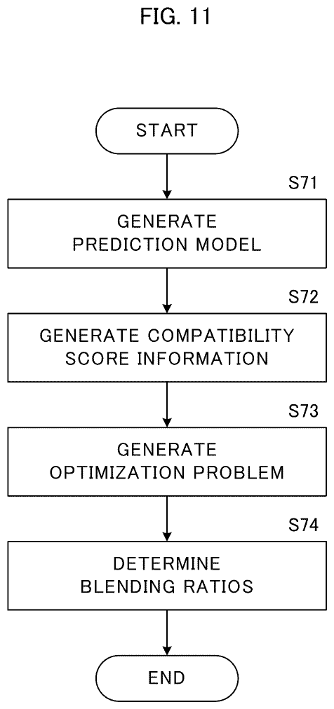

FIG. 11 is a flowchart of a process performed by the blending ratio determination device 70 according to the second example embodiment. The training means 71 performs training using the compatibility scores between the materials whose compatibility is known, and generates a prediction model that predicts the compatibility scores between the materials whose compatibility is unknown (step S71). The compatibility prediction means 72 generates, by using the prediction model, compatibility score information including compatibility scores between the materials whose compatibility is unknown (step S72). The problem generation means 73 generates the optimization problem that includes the compatibility scores between the plurality of materials to be blended in the objective function (step S73). The blending ratio determination means 74 determines the blending ratios of the plurality of materials by solving the optimization problem (step S74). Then, the process is terminated.

According to the blending ratio determination device 70 of the second example embodiment, it is possible to determine the optimum blending ratio considering the compatibility between the materials used.

A part or all of the example embodiments described above may also be described as the following supplementary notes, but not limited thereto.

### (Supplementary Note 1)

A blending ratio determination device comprising:
a training means configured to perform training by using a compatibility score between materials whose compatibility is known, and generate a prediction model that predicts a compatibility score between materials whose compatibility is unknown;
a compatibility prediction means configured to generate, by using the prediction model, compatibility score information including the compatibility score between the materials whose compatibility is unknown;
a problem generation means configured to generate an optimization problem including compatibility scores between a plurality of materials to be blended in an objective function; and
a blending ratio determination means configured to determine blending ratios of the plurality of materials to be blended by solving the optimization problem.

### (Supplementary Note 2)

The blending ratio determination device according to supplementary note 1, wherein the training means generates a graph link indicating the compatibility score between the materials whose compatibility is known, and generates the prediction model by the training using the graph link.

### (Supplementary Note 3)

The blending ratio determination device according to supplementary note 1, wherein the problem generation means sets, as the objective function, a function that adds, at a predetermined ratio, a total nutritional value score that is a sum of nutritional value scores of the plurality of materials to be blended and a total compatibility score that is a sum of the compatibility scores between the plurality of materials to be blended.

### (Supplementary Note 4)

The blending ratio determination device according to supplementary note 3, wherein the problem generation means calculates, for each of pairs obtained by selecting two materials from the plurality of materials to be blended, a product of a blending ratio of each of the two materials and a compatibility score between the two materials, and sets a value obtained by totaling the obtained products for all of the pairs as the total compatibility score.

### (Supplementary Note 5)

The blending ratio determination device according to supplementary note 4, wherein the problem generation means calculates the total nutritional value score by using a table indicating a nutritional value score per unit weight for each material.

### (Supplementary Note 6)

The blending ratio determination device according to supplementary note 5, wherein
the problem generation means sets a value obtained by subtracting a sum of costs of the materials to be blended from the sum of the nutritional value scores of the plurality of materials to be blended as the total nutritional value score, and
the costs include at least one of calories and prices of the materials.

### (Supplementary Note 7)

The blending ratio determination device according to supplementary note 3, wherein the optimization problem includes constraint conditions regarding the number and the nutritional value scores of the materials to be blended.

### (Supplementary Note 8)

A blending ratio determination method executed by a computer, the blending ratio determination method comprising:
performing training by using a compatibility score between materials whose compatibility is known, and generating a prediction model that predicts a compatibility score between materials whose compatibility is unknown;
generating, by using the prediction model, compatibility score information including the compatibility score between the materials whose compatibility is unknown;
generating an optimization problem including compatibility scores between a plurality of materials to be blended in an objective function; and
determining blending ratios of the plurality of materials to be blended by solving the optimization problem.

### (Supplementary Note 9)

A recording medium storing a program, the program causing a computer to perform a process comprising:
performing training by using a compatibility score between materials whose compatibility is known, and generating a prediction model that predicts a compatibility score between materials whose compatibility is unknown;
generating, by using the prediction model, compatibility score information including the compatibility score between the materials whose compatibility is unknown;
generating an optimization problem including compatibility scores between a plurality of materials to be blended in an objective function; and
determining blending ratios of the plurality of materials to be blended by solving the optimization problem.

While the disclosure has been described with reference to the example embodiments and examples, the disclosure is not limited to the above example embodiments and examples. It will be understood by those of ordinary skill in the art that various changes in form and details may be made therein without departing from the spirit and scope of the present disclosure as defined by the claims.

### DESCRIPTION OF SYMBOLS

13 Processor
21 Data acquisition unit
22 Prediction model training unit
23 Compatibility prediction unit
24 Optimization problem generation unit
25 Blending ratio determination unit
100 Blending ratio determination device

What is claimed is:

1. A blending ratio determination device comprising:
at least one memory configured to store instructions; and
at least one processor configured to execute the instructions to:
perform training by machine learning with a compatibility score between materials whose compatibility is known, and generate a prediction model that predicts a compatibility score between materials whose compatibility is unknown;
generate, by using the prediction model, compatibility score information including the compatibility score between the materials whose compatibility is unknown;
generate an optimization problem including compatibility scores between a plurality of materials to be blended in an objective function; and
determine blending ratios of the plurality of materials to be blended by solving the optimization problem.

2. The blending ratio determination device according to claim 1, wherein the processor generates a graph link indicating the compatibility score between the materials whose compatibility is known, and generates the prediction model by the training using the graph link.

3. The blending ratio determination device according to claim 1, wherein the processor sets, as the objective function, a function that adds, at a predetermined ratio, a total nutritional value score that is a sum of nutritional value scores of the plurality of materials to be blended and a total compatibility score that is a sum of the compatibility scores between the plurality of materials to be blended.

4. The blending ratio determination device according to claim 3, wherein the processor calculates, for each of pairs obtained by selecting two materials from the plurality of materials to be blended, a product of a blending ratio of each of the two materials and a compatibility score between the two materials, and sets a value obtained by totaling the obtained products for all of the pairs as the total compatibility score.

5. The blending ratio determination device according to claim 4, wherein the processor calculates the total nutritional value score by using a table indicating a nutritional value score per unit weight for each material.

6. The blending ratio determination device according to claim 5, wherein
the processor sets a value obtained by subtracting a sum of costs of the materials to be blended from the sum of the nutritional value scores of the plurality of materials to be blended as the total nutritional value score, and
the costs include at least one of calories and prices of the materials.

7. The blending ratio determination device according to claim 3, wherein the optimization problem includes constraint conditions regarding the number and the nutritional value scores of the materials to be blended.

8. A blending ratio determination method executed by a computer, the blending ratio determination method comprising:
performing training by machine learning with a compatibility score between materials whose compatibility is known, and generating a prediction model that predicts a compatibility score between materials whose compatibility is unknown;
generating, by using the prediction model, compatibility score information including the compatibility score between the materials whose compatibility is unknown;
generating an optimization problem including compatibility scores between a plurality of materials to be blended in an objective function; and
determining blending ratios of the plurality of materials to be blended by solving the optimization problem.

9. A non-transitory computer readable recording medium storing a program, the program causing a computer to perform a process comprising:
performing training by machine learning with a compatibility score between materials whose compatibility is known, and generating a prediction model that predicts a compatibility score between materials whose compatibility is unknown;
generating, by using the prediction model, compatibility score information including the compatibility score between the materials whose compatibility is unknown;
generating an optimization problem including compatibility scores between a plurality of materials to be blended in an objective function; and
determining blending ratios of the plurality of materials to be blended by solving the optimization problem.
